(19) United States
(12) Patent Application Publication
(10) Pub. No.: US 2018/0014799 A1
(43) Pub. Date: Jan. 18, 2018

(54) SYSTEM CONFIGURED TO HOLD A RADIATION SENSING DEVICE IN A VARIETY OF POSITIONS

(71) Applicants: Harold K. Schmulenson, Scottsdale, AZ (US); Thomas Gillen, Orland Park, IL (US)

(72) Inventors: Harold K. Schmulenson, Scottsdale, AZ (US); Thomas Gillen, Orland Park, IL (US)

(21) Appl. No.: 15/645,089

(22) Filed: Jul. 10, 2017

Related U.S. Application Data
(60) Provisional application No. 62/363,457, filed on Jul. 18, 2016.

(51) Int. Cl. A61B 6/14; A61B 6/00

(52) U.S. Cl. CPC A61B 6/145; A61B 6/4283

(57) ABSTRACT

A system is provided for use during a medical procedure. The system has at least one sensing device holder configured to hold a radiation sensing device, a ring guide, and a rod which connects the holder to the ring guide. The ring guide includes a ring having a central opening and first and second connecting portions extending outwardly therefrom. The ring defines horizontal and vertical axes which pass through an axial centerline of the ring guide. Each connecting portion has a channel which accepts the rod therethrough and which is parallel to the axial centerline. The channel of the first connecting portion intersects the horizontal axis, and is offset from the vertical axis. The channel of the second connecting portion is offset from the horizontal and vertical axes. The rod can be attached to either of the connecting portions.

### CROSS-REFERENCE TO RELATED APPLICATION

This application claims the domestic benefit of U.S. provisional application Ser. No. 62/363,457, filed on Jul. 18, 2016, the contents of which are incorporated herein in its entirety.

### FIELD OF THE DISCLOSURE

This disclosure relates generally to a system that holds all dental media such as X-ray films, dental sensors and Phosphor Storage Plates (PSP) plates.

### BACKGROUND

Dental radiographs are made using x-ray examination units, often including an x-ray cone or tube positioned proximate the patient and aligned to take x-rays of certain teeth. Dental x-ray sensing devices, which include including x-ray film units, digital x-ray sensors, charge coupled devices, phosphor imaging plates or the like, often have a generally flat or plate-like configuration and standardized dimensions so that the sensing device can be placed into the oral cavity.

The sensing device is placed into the patient's mouth and held in place proximate to the tooth or teeth to be examined. The x-rays are directed through the target teeth to the sensor. It has been found that proper orientation of the sensor is required to eliminate distortions and improper focus.

To ensure proper orientation of the sensing device, sensor carriers or sensing device holders with “bite blocks” have been developed. These devices often have a plate for holding the sensing device and a bite block that the patient bites down upon to position the device and the carried sensor. A bite block is shown for example, in U.S. Pat. No. 3,473,026.

Different sensing devices are often used depending upon the area of the mouth to be examined. This may include for example, endo, posterior, anterior, left, right, upper and lower bite wings, and the like. Known bite blocks and sensing device holders have been individually designed and manufactured for each different type of sensing device. The dimensions of the sensing device and the sensing device holder dictate the degree of secured positioning of the sensing device in the sensing device holder.

A dental professional may have a large number of x-ray sensing devices with varying sizes and shapes, and hence, a similarly large number of sensing device holders. The dental professional is often faced with employing a different sensing device or set of sensing devices, sensing device holders and bite blocks depending upon the particular x-ray procedure being employed and the area of the mouth to be examined. At best, it is time consuming to change between sensing devices, sensing device holders and bite blocks.

In order to precisely align the x-ray cone or tube with a particular x-ray sensing device held by a particular sensing device holder, a rod and ring guide combination may be employed. The rod is typically attached to a particular sensing device holder at one end and connected with the ring guide at the other end. The ring guide helps to aim the x-ray cone or tube at the x-ray sensing device. However, some times, in order to take x-rays of various different portions of the mouth, multiple sensing device holders, bite blocks, rods and rings may need to be combined with each other to form a particular rod and ring guide combination.

### SUMMARY

A system provided for use during a medical procedure in accordance with some example embodiments includes at least one sensing device holder configured to hold a radiation sensing device, a ring guide, and a rod which connects the holder to the ring guide. The ring guide includes a ring having a central opening and first and second connecting portions extending outwardly therefrom. The ring defines horizontal and vertical axes which pass through an axial centerline of the ring guide. Each connecting portion has a channel which accepts the rod therethrough and which is parallel to the axial centerline. The channel of the first connecting portion intersects the horizontal axis, and is offset from the vertical axis. The channel of the second connecting portion is offset from the horizontal and vertical axes. The rod can be attached to either of the connecting portions.

This Summary is provided merely for purposes of summarizing some example embodiments so as to provide a basic understanding of some aspects of the disclosure. Accordingly, it will be appreciated that the above described example embodiments are merely examples and should not be construed to narrow the scope or spirit of the disclosure in any way. Other embodiments, aspects, and advantages of various disclosed embodiments will become apparent from the following detailed description taken in conjunction with the accompanying drawings which illustrate, by way of example, the principles of the described embodiments.

### BRIEF DESCRIPTION OF THE DRAWINGS

The organization and manner of the structure and operation of the disclosed embodiments, together with further objects and advantages thereof, may best be understood by reference to the following description, taken in connection with the accompanying drawings, which are not necessarily drawn to scale, wherein like reference numerals identify like elements in which:

FIG. 1 is a side elevation view of a system which includes a sensing device holder, a ring guide and a rod;

FIG. 2 is an end plan view of the rod of FIG. 1;

FIG. 3 is a side elevation view of the sensing device holder of FIG. 1;

FIG. 4 is a front perspective view of the sensing device holder;

FIG. 5 is a rear perspective view of the sensing device holder shown holding a prior art radiation sensing device;

FIG. 6 is a front elevation view of the sensing device holder;

FIG. 7 is a rear elevation view of the sensing device holder;

FIG. 8 is a perspective view of a prior art radiation sensing device;

FIG. 9 is a first side elevation view of the ring guide of FIG. 1;

FIG. 10 is a second side elevation view of the ring guide;

FIG. 11 is a perspective view of the ring guide;

FIG. 12 is a front elevation view of the ring guide;

FIG. 13 is a side elevation view of the patient showing the ring guide oriented for use in taking an upper right posterior image;

FIG. 14 is a side elevation view of the patient showing the ring guide oriented for use in taking a lower right posterior image;

FIG. 15 is a side elevation view of the patient showing the ring guide oriented for use in taking an upper left posterior image;

FIG. 16 is a side elevation view of the patient showing the ring guide oriented for use in taking a lower left posterior image;

FIG. 17 is a side elevation view of the patient showing the ring guide oriented for use in taking an upper anterior image;

FIG. 18 is a side elevation view of the patient showing the ring guide oriented for use in taking a lower anterior image;

FIG. 19 is a side elevation view of the patient showing the ring guide oriented for use in taking bitewing right side image;

FIG. 20 is a side elevation view of the patient showing the ring guide oriented for use in taking a bitewing left side image;

FIG. 21 is a rear perspective view showing the sensing device holder holding the prior art radiation sensing device in a first horizontal position;

FIG. 22 is a rear perspective view showing the sensing device holder holding the prior art radiation sensing device in a second horizontal position;

FIG. 23 is a front perspective view showing the sensing device holder holding the prior art radiation sensing device in the second horizontal position;

FIG. 24 is a rear perspective view showing a modified sensing device holder holding the prior art radiation sensing device in a first vertical position;

FIG. 25 is a rear perspective view showing the sensing device holder of FIG. 24 holding the prior art radiation sensing device in a second vertical position; and

FIG. 26 is a front perspective view showing the sensing device holder of FIG. 24 holding the prior art radiation sensing device in the second vertical position.

### DETAILED DESCRIPTION

While the disclosure may be susceptible to embodiment in different forms, there is shown in the drawings, and herein will be described in detail, specific embodiments with the understanding that the present disclosure is to be considered an exemplification of the principles of the disclosure, and is not intended to limit the disclosure to that as illustrated and described herein. Therefore, unless otherwise noted, features disclosed herein may be combined together to form additional combinations that were not otherwise shown for purposes of brevity.

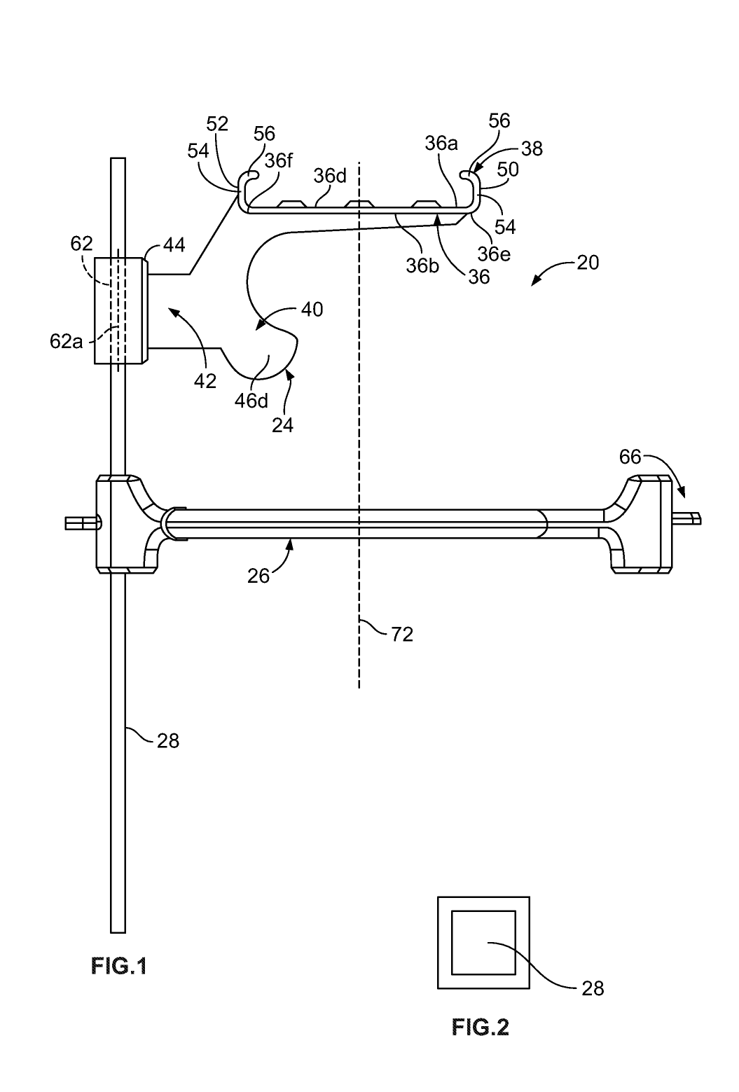

A system 20, see FIG. 1, is provided for holding a radiation sensing device 22, see FIG. 7, for taking x-rays of a patient's mouth during a medical procedure, such as during a dental, endodontic or orthodontic procedure. The system 20 includes at least one sensing device holder 24 which holds the radiation sensing device 22 and which is configured to be attached to a ring guide 26 by a rod 28. The sensing device holder 24 can be attached to the ring guide 26 by the rod 28 at two different locations on the ring guide 26. The ring guide 26 is used to precisely aim a radiation generating machine, such as an x-ray machine, at and direct radiation from the radiation generating machine to the radiation sensing device 22. The ring guide 26 aims and aligns a cone of the radiation generating machine with the radiation sensing device 22. The rod 28 is slideably connected with the ring guide 26 to allow the distance between the ring guide 24 and the radiation sensing device 22 to be varied.

The radiation sensing device 22 is any device which can be used to sense radiation, such as gamma wave radiation, light wave radiation and, preferably, x-ray radiation. As best shown in FIG. 7, the radiation sensing device 22 preferably includes a housing 30 which surrounds either a radiation film unit or a radiation sensor unit 32. The radiation film unit uses film to detect radiation, such as x-ray radiation. The radiation sensor unit 32 uses a digital sensor or a charge coupled device to detect radiation such as x-ray films, dental sensors and Phosphor Storage Plates (PSP) plates or the like. Radiation sensor unit may include a wire 34 which is used to provide power and/or transfer signals between the digital radiation sensor and a control unit, not shown. Preferably, radiation sensing device 22 is a dental x-ray sensing device which is sized for use in the mouth of a patient in order to take x-ray scans of a patient's teeth. Radiation sensing devices 22 can vary in width, height and thickness. Preferably the width of the radiation sensing device 22 is between 3 and 8 centimeters. Also, preferably the height of the radiation sensing device 22 is between 1 and 4 centimeters and the thickness is preferably between 0.1 and 20 millimeters, and more preferably, between 1 to 10 millimeters.

As shown, the housing 30 is rectangular having front and back surfaces 30a, 30b, side surfaces 30c, 30d, 30e, 30f extending between the front and back surfaces 30a, 30b. The housing 30 may take other shapes, such as round or square. Preferably, the housing 30 completely envelopes the radiation film unit or the radiation sensor unit 32, however, the housing 30 may have a window, or a plurality of windows exposing a portion of the radiation film unit or the radiation sensor unit 32. Preferably, the housing 30 is manufactured using an injection molded process in order to reduce costs, however, the housing 30 can be manufactured in one of many ways. For example, housing 30 may be machined, thermoformed, and hand-made. Preferably, in order to reduce costs and maintain rigidity, housing 30 is a one-piece unit which is integrally formed, or a two-piece unit which is snap-fit together. However, housing 30 may comprise multiple parts which are then assembled and fitted together. Preferably, housing 30 is constructed from a rigid yet somewhat flexible material through which radiation can pass, such as but not limited to: metals such as iron, steel, stainless steel, aluminum, silver, titanium, and brass; plastics, such as ethylene, vinyl, acetate; acrylics, such as acrylonitrol-butadine-styrene; resins; and polymers such as polycarbonate. Preferably, the housing 30 is formed from polyphenylsulfone (PPSU) or polypropylene. The housing 30 may be colored any one of various different colors depending on the size and type of sensors used. For example, the housing 30 may be colored white for a size two x-ray film unit or colored green for a size zero x-ray film unit.

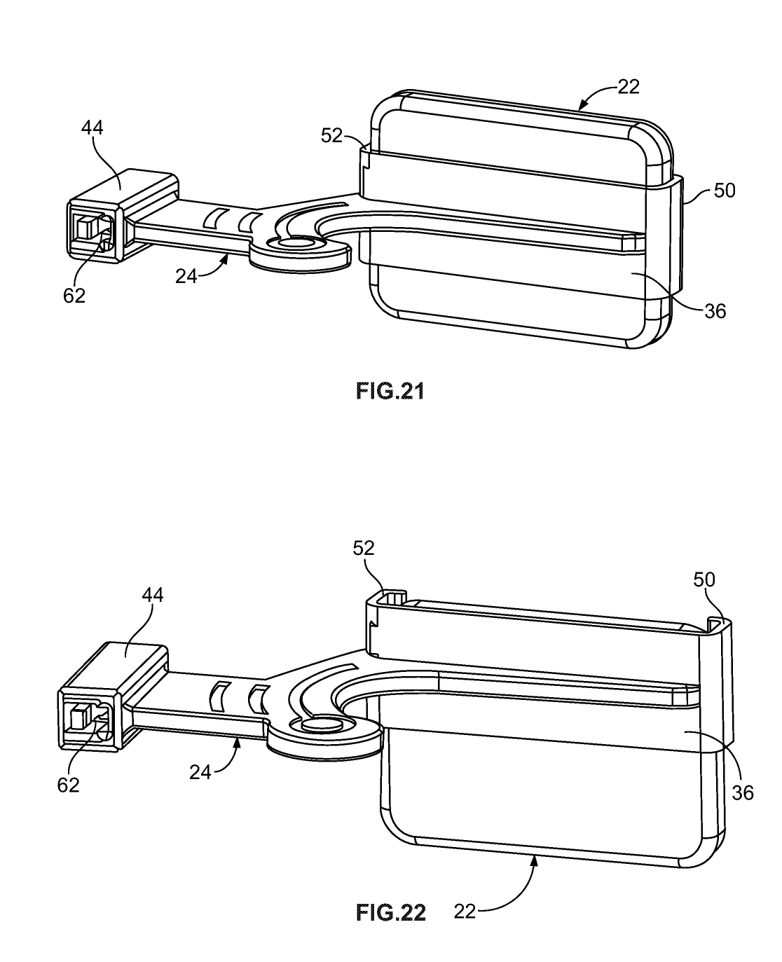

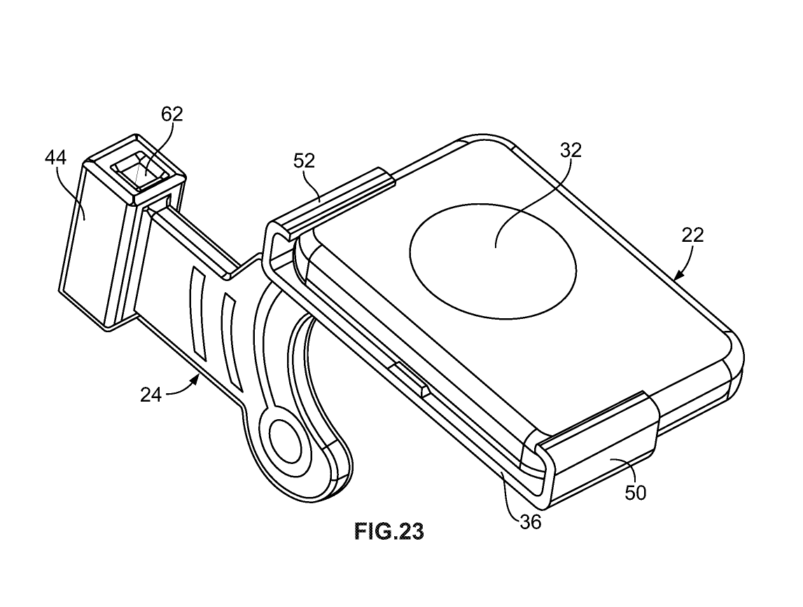

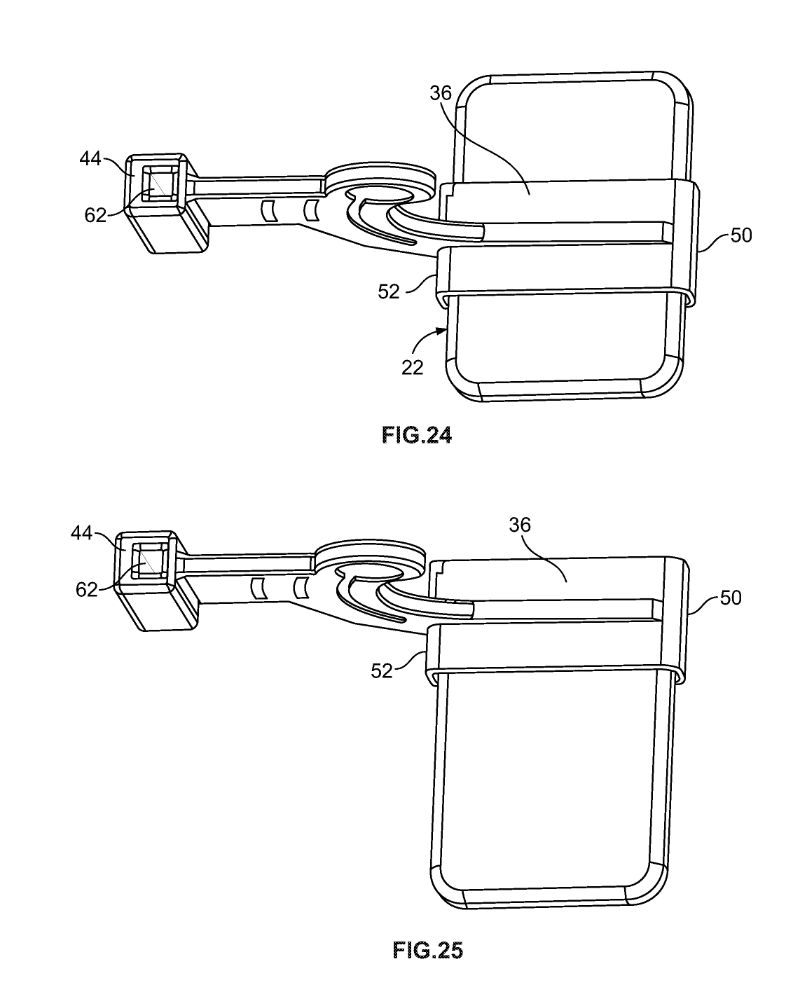

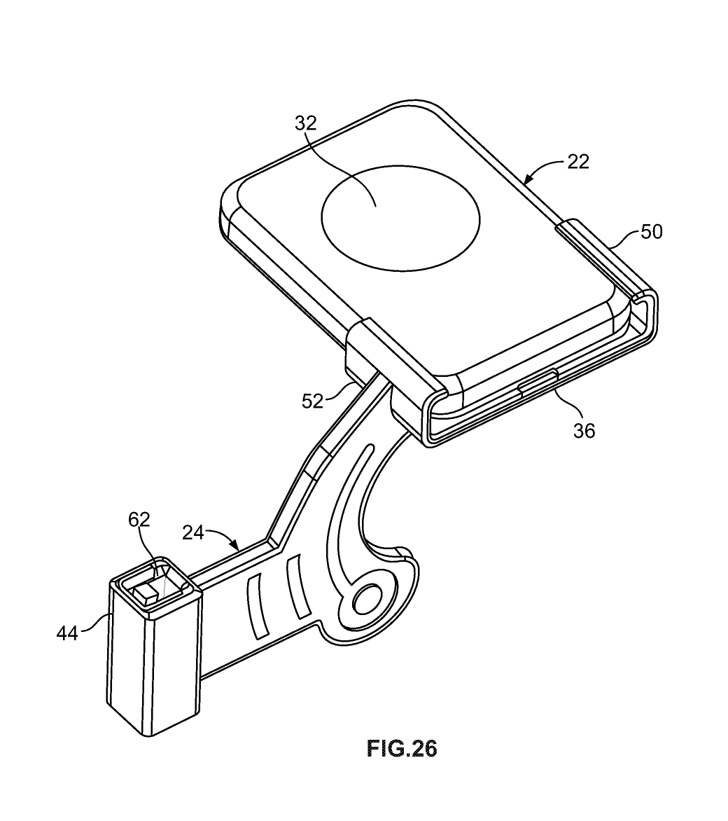

The sensing device holder 24 is designed to hold and retain the radiation sensing device 22 in a variety of positions as shown in FIGS. 21-26. Preferably, the sensing device holder 24 is manufactured using an injection molded process in order to reduce costs, however, the sensing device holder 24 can be manufactured in one of many ways. For example, the sensing device holder 24 may be machined, thermoformed, and hand-made. Preferably, sensing device holder 24 is constructed from a rigid yet somewhat flexible material, such as but not limited to: metals such as iron, steel, stainless steel, aluminum, silver, titanium, and brass; plastics, such as ethylene, vinyl, acetate; acrylics, such as acrylonitrol-butadine-styrene; resins; and polymers such as polycarbonate. Preferably, the sensing device holder 24 is formed from polyphenylsulfone (PPSU) or polypropylene. The sensing device holder 24 may be colored any one of various different colors depending on the size and type of sensors used. For example, the sensing device holder 24 may be colored white for a size two x-ray film unit or colored green for a size zero x-ray film unit.

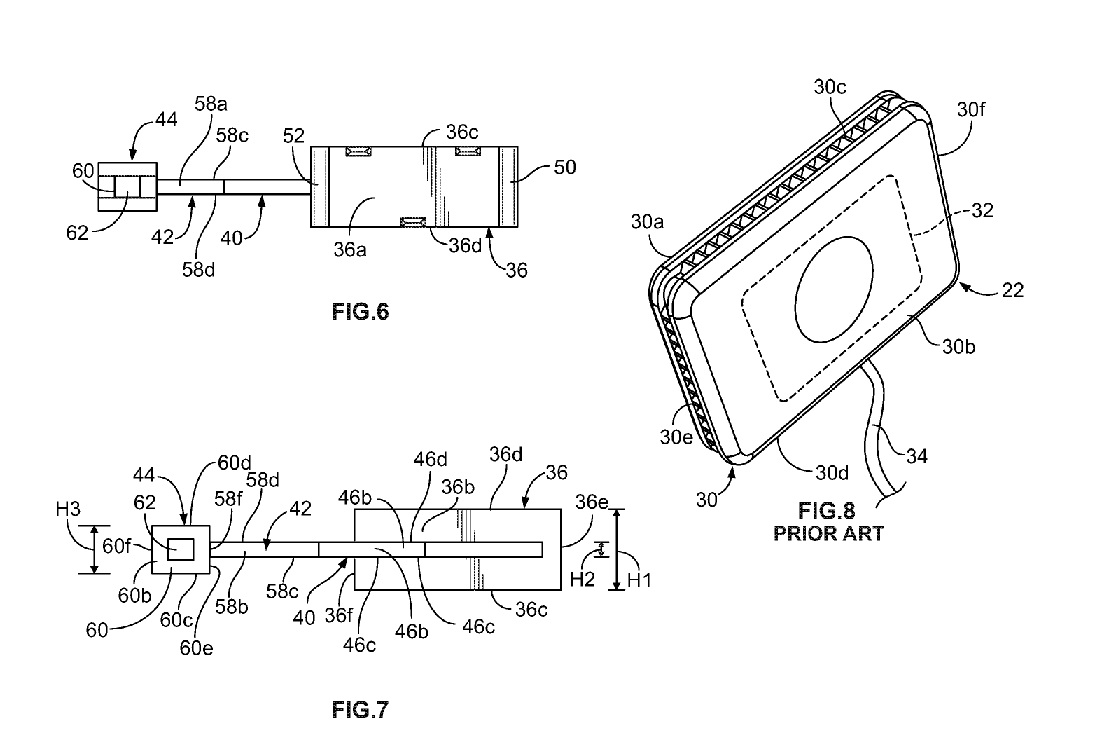

As best shown in FIGS. 2-7, the sensing device holder 24 includes an elongated support member 36, a retention assembly 38 attached to a front side 36a of the support member 36, a bite block 40 attached to and extending from a back side 36b of the support member 36, a bridge 42 extending from the bite block 40 to a mount 44 which is attached to the rod 28. The sensing device holder 24 is a one-piece unit which is integrally formed and may be formed by injection molding. The retention assembly 38 is used to hold the radiation sensing device 22 as described herein. A patient bites onto the bite block 40 to hold the sensing device holder 24 in the mouth of the patient in different locations within the mouth. The bridge 42 extends through the lips of the patient and outwardly from the patient's mouth. The mount 44 is located outside of the patient's mouth.

The support member 36 is planar and has front and back sides 36a, 36b, a first surface 36c extending between the front and back sides 36a, 36b, a second surface 36d extending between the front and back sides 36a, 36b, a leading end 36e and a trailing end 36f. The distance between the first surface 36c and the second surface 36d defines a height H1 of the support member 36. A midpoint 36g of the support member 36 is provided half way between the leading end 36e and the trailing end 36f.

The bite block 40 extends from the back side 36b of the support member 36. The bite block 40 shown in the drawings is exemplary and a variety of shapes of bite blocks can be used. The bite block 40 may be planar, may have a variety of surface features thereon which allow the patient to better grip the bite block 40 with his/her teeth.

As shown in the exemplary bite block 40, the bite block 40 is formed of a body 46 having front and back surfaces 46a, 46b, a first surface 46c extending between the front and back surfaces 46a, 46b, a second surface 46d extending between the front and back surfaces 46a, 46b, a leading end 46e and a trailing end 46f. A first portion of the front surface 46a is integrally formed with the back side 36b of the support member 36; a second portion of the front surface 46a extends from the back side 36b of the support member 36. The first and second surfaces 46c, 46d form upper and lower surfaces depending upon which side of the mouth the sensing device holder 24 is used. The distance between the first and second surfaces 46c, 46d defines a height H2 of the bite block 40. The bite block 40 preferably has a height H2 which is less than the height H1 of the support member 36. The body 46 may be shaped such that the leading end surface 46e provides a recess 48 which accommodates any structure mounted on a patient's tooth, such as a clamp for a dental dam. This recess 48 may be eliminated and the bite block 40 formed as a square, rectangle or any other desirable shape. When the sensing device holder 24 is inserted into a patient's mouth, the patient is able to bite down with the patient's teeth on the surfaces 46c, 46d of the bite block 40.

The retention assembly 38 includes a leading retention guide 50 and a trailing retention guide 52 extending from the front side 36a of the support member 36. The leading retention guide 50 extends from the front side 36a of the support member 36 proximate to the leading end 36e of the support member 36. The trailing retention guide 52 extends from the front side 36a of the support member 36 at the trailing end 36f of the support member 36. Each retention guide 50, 52 may be formed from a generally L-shaped cross-section having an extending portion 54 which extends perpendicularly relative to the support member 36 and a gripping portion 56 which extends perpendicularly from or curves inwardly toward the extending portion 54 and is parallel to or curves inwardly toward the support member 36. The retention assembly 38 may include structure for allowing retention guide 50, 52 to move relative to each other, for example, plungers and biasing members as is known in the prior art.

The radiation sensing device 22 is mounted between the retention guides 50, 52 in either a horizontal or vertical position as shown in FIGS. 21-26. The retention assembly 38 is appropriately sized to securely hold the radiation sensing device 22 in either the horizontal or vertical position. When the radiation sensing device 22 is mounted between the retention guides 50, 52, the sensor 32 preferably is positioned such that the majority of the sensor 32 is above or below the support member 36. To effect this, the radiation sensing device 22 can be slid relative to the retention guides 50, 52 and the support member 36 such that the surface 30b of the radiation sensing device 22 is proximate to the first surface 36c of the support member 36 or the surface 30d of the radiation sensing device 22 is proximate to the second surface 36d the support member 36. FIG. 5 shows alternate positions of the radiation sensing device 22 in dotted line.

The bridge 42 extends between the bite block 40 and the mount 44. The bridge 42 is elongated such that the bridge 42 is long enough to extend from the bite block 40, through the lips of the patient and outwardly from the mouth of the patient. The bridge 42 may be formed as a rectangle. The bridge 42 is formed of a body 58 having front and back surfaces 58a, 58b, a first surface 58c extending between the front and back surfaces 58a, 58b, a second surface 58d extending between the front and back surfaces 58a, 58b, a leading end 58e and a trailing end 58f The leading end 58e merges with the trailing end 46f of the bite block 40. The front surface 58a merges with the second portion of the front surface 46a of the bite block 40; the back surface 58b merges with the back surface 46b of the bite block 40. The first surface 58c merges with and is planar with the first surface 46c of the bite block 40. The second surface 58d merges with and is planar with the first surface 46d of the bite block 40. The trailing end 58f merges with the mount 44.

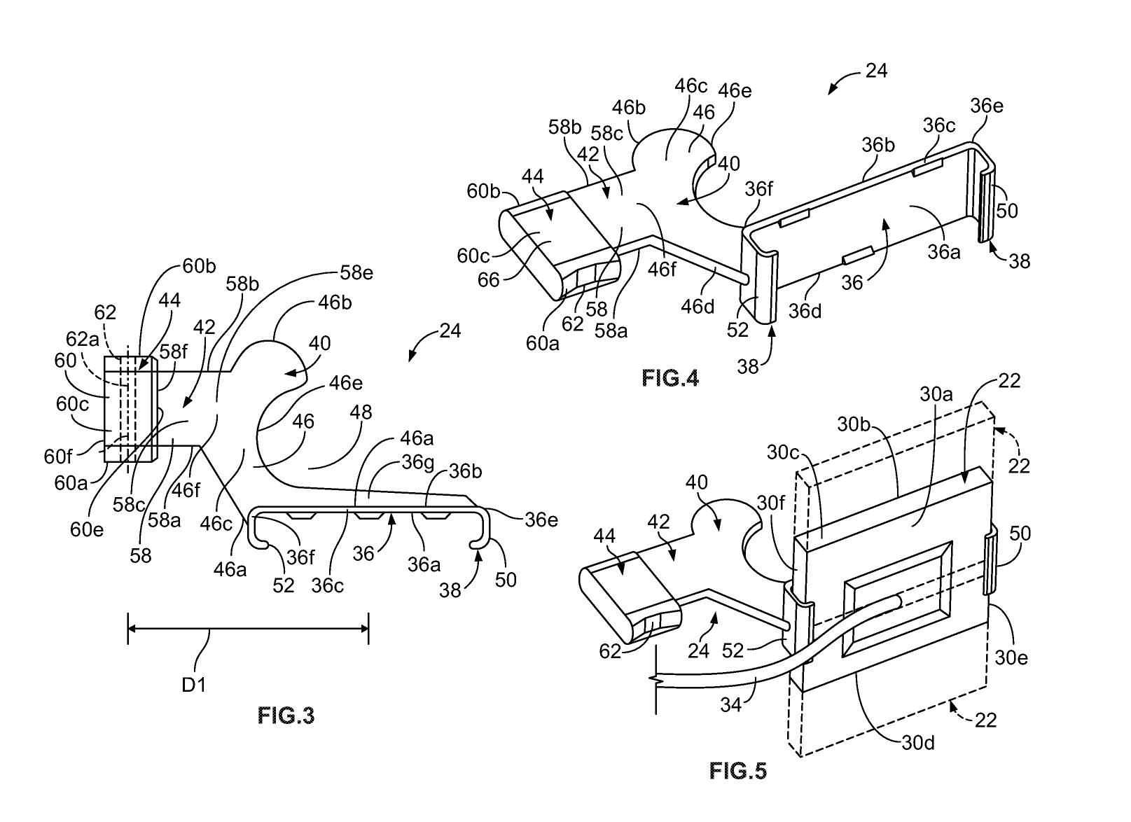

The mount 44 extends from the trailing end 58f of the bridge 42. The mount 44 may be formed as a block. The mount 44 is formed of a body 60 having front and back surfaces 60a, 60b, a first surface 60c extending between the front and back surfaces 60a, 60b, a second surface 60d extending between the front and back surfaces 60a, 60b, a leading end 60e and a trailing end 60f. The leading end 60e is connected to and integrally formed with the trailing end 58f of the bridge 42. The mount 44 has a single channel 62 which extends through the body 60 from the front surface 60a to the back surface 60b into which the rod 28 is slidably accepted. The channel 62 defines an axial centerline 62a, see FIG. 3, therethrough. The channel 62 does not have a circular profile. For example, the channel 62 may be square, rectangular, splined, D-shaped, etc. The distance between the first and second surfaces 46c, 46d defines a height H3 of the mount 44. The height H3 of the mount 44 is greater than the height H2 of the bridge 42 such that the surfaces 60c, 60d of the mount 44 are offset from the surfaces 58c, 58d of the bridge 42.

A distance D1 is defined between the centerline 62a of the channel 62 and the midpoint 36g of the support member 36.

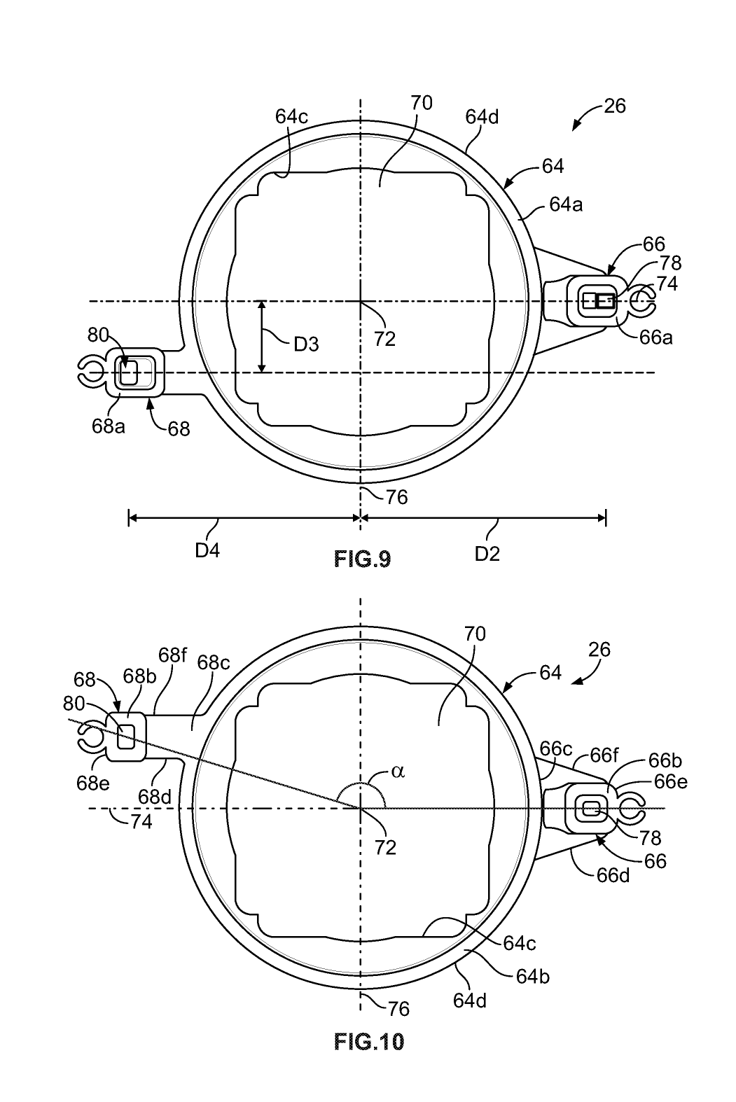

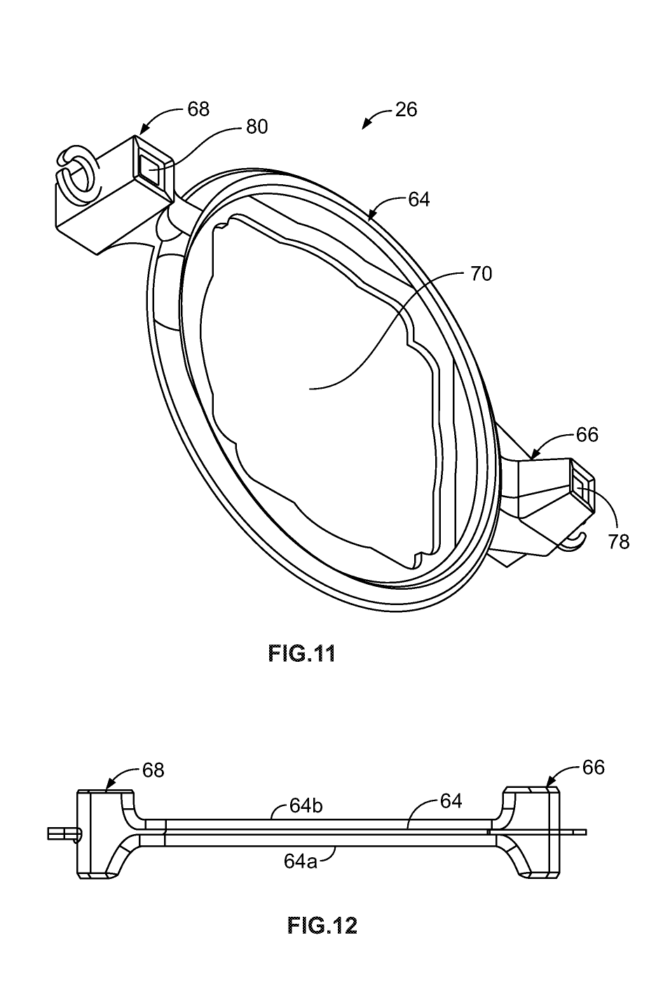

As best shown in FIGS. 9-12, the ring guide 26 is formed of a generally circular ring 64 having first and second surfaces 64a, 64b which define an inner perimeter surface 64c that extends between the first and second surfaces 64a, 64b, and an outer perimeter surface 64d that extends between the first and second surfaces 64a, 64b, a first connecting portion 66 extending outwardly from the outer perimeter 64a, and a second connecting portion 68 extending outwardly from the outer perimeter 64a.

The inner perimeter surface 64c forms an opening 70 in the ring 64. The opening 70 has an axial centerline 72 which extends from the first surface 64a to the second surface 64b as shown at 72. The opening 70 may be non-circular as shown, or may be circular. The ring 64 defines first and second axes 74, 76 which are perpendicular to each other, which pass through the axial centerline 72, and which are co-planar with the first and second surfaces 64a, 64b. As shown in the drawings, axis 74 is a horizontal axis and axis 76 is a vertical axis.

The first connecting portion 66 may be formed as a block having first and second surfaces 66a, 66b and side surfaces 66c, 66d, 66e, 66f extending between the first and second surfaces 66a, 66b. The side surface 66c merges with the outer perimeter surface 64d of the ring 64. A channel 78 extends through the first connecting portion 66 from the first surface 66a to the second surface 66b. The channel 78 does not have a circular profile. For example, the channel 78 may be square, rectangular, splined, D-shaped, etc. The channel 78 intersects the axis 74 and is parallel to the axial centerline 72. The channel 78 is offset from the axis 76 a predetermined distance D2 along the axis 74.

The second connecting portion 68 may be formed as a block having first and second surfaces 68a, 68b and side surfaces 68c, 68d, 68e, 68f extending between the first and second surfaces 68a, 68b. The side surface 68c merges with the outer perimeter surface 64d of the ring 64. A channel 80 extends through the second connecting portion 68 from the first surface 68a to the second surface 68b. The channel 80 does not have a circular profile. For example, the channel 80 may be square, rectangular, splined, D-shaped, etc. The channel 80 is offset from the axis 74 a predetermined distance along the axis 76 by distance D3, and is parallel to the axial centerline 72. The channel 80 is offset from the axis 76 a predetermined distance D4 along the axis 74.

The channel 78 is on one side of the axis 76 and the channel 80 is on the other side of the axis 76 such that the channels 78, 80 are generally diametrically opposed on the ring 64. The channel 78 is radially spaced from the centerline 72 and the channel 78 is radially spaced from the centerline 72. The channel 80 is spaced from the channel 78 around the circumference of the ring 64 by an obtuse angle α, see FIG. 10. In an embodiment, the angle is 157.5 degrees.

In a preferred embodiment, D2 and D4 are equal.

The rod 28 is linear, and has a non-circular cross-section. The rod 28 is composed of a rigid material such as a plastic or metal, for example aluminum, steel, or nickel. The cross-section of the rod 28 is complementary to the shape of the channels 62, 78, 80 through the mount 44 and the first and second connection portions 66, 68 such that when the rod 28 is inserted into any of the channels 62, 78, 80, the rod 28 does not rotate relative to the mount 44 and to the first and second connection portions 66, 68. If desired, the rod 28 can have a first non-circular cross-section at a first end which is complementary to and mates with the channel 62 of the mount 44, and the rod 28 can have a second non-circular cross-section at a second end which is complementary to and mates with the channels 78, 80 of the first and second connection portions 66, 68, the first and second ends having different cross-sections. In some embodiments, the rod 28 has two projections (not shown), such as those shown in U.S. Pat. No. 9,354,506, the contents of which are incorporated herein by reference, which are configured to attach to the sensing device holder 24. In some embodiments, the rod 28 has an adapter having the two conventional projections (not shown), such as those shown in U.S. Pat. No. 9,354,50, attached thereto configured to attach to the sensing device holder 24. In these embodiments, the rod 28 would not extend through the channel of the mount 44 of the sensing device holder 24 as discussed herein.

In use, the rod 28 is inserted into the channel 62 of the mount 44 of the sensing device holder 24 and into one of the first and second connection portions 66, 68 of the ring guide 26. When the rod 28 is inserted into the first connecting portion 66 of the ring guide 26, the midpoint 36g of the sensor holder 24 is aligned with the axial centerline 72 and bite wing x-rays may be taken as described herein. When the rod 28 is inserted into the second connecting portion 68 of the ring guide 26, the midpoint 36g of the sensor holder 24 is offset from, but parallel to, the axial centerline 72 and an anterior or posterior x-ray may be taken may be taken as described herein. Thus, the same three components, the sensing device holder 24, the rod 28 and the ring guide 26, are used to image all portions of the patient's mouth. The three components, the sensing device holder 24, the rod 28 and the ring guide 26, can be easily dissembled from each other, and reassembled into the alternate positions.

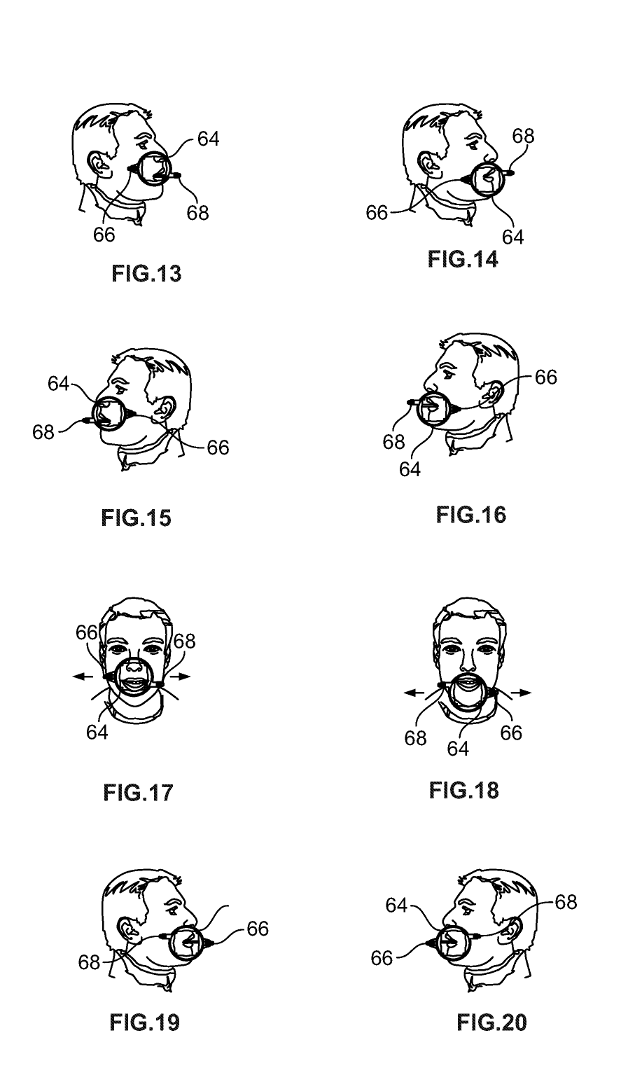

The ring guide 26 can be placed in a variety of positions relative to the patient as shown in FIGS. 13-20.

To take an upper right posterior image as shown in FIG. 13, the ring guide 26 is positioned such that the first surface 64a faces and overlaps the right cheek of the patient and the second connecting portion 68 extends forwardly of the lips of the patient. When looking at the patient from the right side, the second connecting portion 68 is at the four o'clock position and the first connecting portion 66 is at the nine o'clock position. The rod 28 is attached to the second connecting portion 68 and to the sensing device holder 24. The support member 36 overlaps the opening 70, and the back side 36b of the sensing device holder 24 faces the first surface 64a of the ring guide 26. The radiation sensing device 22 is inserted into the retention assembly 38 such that the surface 30d of the radiation sensing device 22 is aligned with the second surface 36d of the retention assembly 38. During insertion of the sensing device holder 24 into the patient's mouth, the sensing device holder 24 is inserted with the leading retention guide 50 first going into the mouth followed by the leading end surface 44 of the bite block 40, with the trailing retention guide 52 and the trailing end 46f of the bite block 40 following. The retention assembly 38 and bite block 40 pass over the teeth of the patient and the support member 36, the retention guides 50, 52 and the radiation sensing device 22 are in the interior of the mouth. When the bite block 40 is correctly positioned proximate to the upper right posterior teeth, the patient bites down onto the bite block 40 with his/her right posterior teeth to securely hold the sensing device holder 24 in place. The bridge 42 extends outwardly from the teeth and through the lips of the patient. The mount 44 and rod 28 are exterior to the lips. The ring guide 26 overlaps the right cheek of the patient and the first surface 64a of the ring guide 26 faces the right cheek of the patient.

To take a lower right posterior image as shown in FIG. 14, the ring guide 26 is positioned such that the second surface 64b faces and overlaps the right cheek of the patient and the second connecting portion 68 extends forwardly of the lips of the patient. When looking at the patient from the right side, the second connecting portion 68 is at the two o'clock position and the first connecting portion 66 is at the nine o'clock position. The rod 28 is attached to the second connecting portion 68 and to the sensing device holder 24. The support member 36 overlaps the opening 70, and the back side 36b of the sensing device holder 24 faces the second surface 64b of the ring guide 26. The radiation sensing device 22 is inserted into the retention assembly 38 such that the surface 30c of the radiation sensing device 22 is aligned with the first surface 36c of the retention assembly 38. During insertion of the sensing device holder 24 into the patient's mouth, the sensing device holder 24 is inserted with the leading retention guide 50 first going into the mouth followed by the leading end surface 44 of the bite block 40, with the trailing retention guide 52 and the trailing end 46f of the bite block 40 following. The retention assembly 38 and bite block 40 pass over the teeth of the patient and the support member 36, the retention guides 50, 52 and the radiation sensing device 22 are in the interior of the mouth. When the bite block 40 is correctly positioned proximate to the lower right posterior teeth, the patient bites down onto the bite block 40 with his/her right posterior teeth to securely hold the sensing device holder 24 in place. The bridge 42 extends outwardly from the teeth and through the lips of the patient. The mount 44 and rod 28 are exterior to the lips. The ring guide 26 overlaps the right cheek of the patient and the second surface 64b of the ring guide 26 faces the right cheek of the patient.

To take an upper left posterior image as shown in FIG. 15, the ring guide 26 is positioned such that the second surface 64b faces and overlaps the left cheek of the patient and the second connecting portion 68 extends forwardly of the lips of the patient. When looking at the patient from the left side, the second connecting portion 68 is at the eight o'clock position and the first connecting portion 66 is at the three o'clock position. The rod 28 is attached to the second connecting portion 68 and to the sensing device holder 24. The support member 36 overlaps the opening 70, and the back side 36b of the sensing device holder 24 faces the second surface 64b of the ring guide 26. The radiation sensing device 22 is inserted into the retention assembly 38 such that the surface 30c of the radiation sensing device 22 is aligned with the first surface 36c of the retention assembly 38. During insertion of the sensing device holder 24 into the patient's mouth, the sensing device holder 24 is inserted with the leading retention guide 50 first going into the mouth followed by the leading end surface 44 of the bite block 40, with the trailing retention guide 52 and the trailing end 46f of the bite block 40 following. The retention assembly 38 and bite block 40 pass over the teeth of the patient and the support member 36, the retention guides 50, 52 and the radiation sensing device 22 are in the interior of the mouth. When the bite block 40 is correctly positioned proximate to the upper left posterior teeth, the patient bites down onto the bite block 40 with his/her left posterior teeth to securely hold the sensing device holder 24 in place. The bridge 42 extends outwardly from the teeth and through the lips of the patient. The mount 44 and rod 28 are exterior to the lips. The ring guide 26 overlaps the left cheek of the patient and the second surface 64b of the ring guide 26 faces the left cheek of the patient.

To take a lower left posterior image as shown in FIG. 16, the ring guide 26 is positioned such that the first surface 64a faces and overlaps the left cheek of the patient and the second connecting portion 68 extends forwardly of the lips of the patient. When looking at the patient from the left side, the second connecting portion 68 is at the ten o'clock position and the first connecting portion 66 is at the three o'clock position. The rod 28 is attached to the second connecting portion 68 and to the sensing device holder 24. The support member 36 overlaps the opening 70, and the back side 36b faces the first surface 64a of the ring guide 26. The radiation sensing device 22 is inserted into the retention assembly 38 such that the surface 30d of the radiation sensing device 22 is aligned with the second surface 36d of the retention assembly 38. During insertion of the sensing device holder 24 into the patient's mouth, the sensing device holder 24 is inserted with the leading retention guide 50 first going into the mouth followed by the leading end surface 44 of the bite block 40, with the trailing retention guide 52 and the trailing end 46f of the bite block 40 following. The retention assembly 38 and bite block 40 pass over the teeth of the patient and the support member 36, the retention guides 50, 52 and the radiation sensing device 22 are in the interior of the mouth. When the bite block 40 is correctly positioned proximate to the lower left posterior teeth, the patient bites down onto the bite block 40 with his/her left posterior teeth to securely hold the sensing device holder 24 in place. The bridge 42 extends outwardly from the teeth and through the lips of the patient. The mount 44 and rod 28 are exterior to the lips. The ring guide 26 overlaps the left cheek of the patient and the first surface 64a of the ring guide 26 faces the left cheek of the patient.

To take an upper anterior image as shown in FIG. 17, the ring guide 26 is positioned such that the first surface 64a faces and overlaps the lips of the patient. When looking at the patient from the front of the face, the second connecting portion 68 is at the four o'clock position and overlap the front of the left cheek, and the first connecting portion 66 is at the nine o'clock position and overlaps the front of the right cheek. The rod 28 is attached to the second connecting portion 68 and to the sensing device holder 24. The support member 36 overlaps the opening 70, and the back side 36b faces the first surface 64a of the ring guide 26. The radiation sensing device 22 is inserted into the retention assembly 38 such that the surface 30d of the radiation sensing device 22 is aligned with the second surface 36d of the retention assembly 38. During insertion of the sensing device holder 24 into the patient's mouth, the sensing device holder 24 is inserted with the retention guides 50, 52 first going into the mouth followed by the front surface 46a of the bite block 40, with the back surface 46b following. The retention assembly 38 and bite block 40 pass over the teeth of the patient and the support member 36, the retention guides 50, 52 and the radiation sensing device 22 are in the interior of the mouth. When the bite block 40 is correctly positioned proximate to the anterior teeth, the patient bites down onto the bite block 40 with his/her anterior teeth to securely hold the sensing device holder 24 in place. The bridge 42 extends outwardly from the teeth and through the lips of the patient. The mount 44 and rod 28 are exterior to the lips. The ring guide 26 overlaps the lips of the patient and the first surface 64a of the ring guide 26 faces the lips of the patient.

To take a lower anterior image as shown in FIG. 18, the ring guide 26 is positioned such that the first surface 64a faces and overlaps the lips of the patient. When looking at the patient from the front of the face, the second connecting portion 68 is at the ten o'clock position and overlaps the front of the right cheek, and the first connecting portion 66 is at the three o'clock position and overlaps the front of the left cheek. The rod 28 is attached to the second connecting portion 68 and to the sensing device holder 24. The support member 36 overlaps the opening 70, and the back side 36b faces the first surface 64a of the ring guide 26. The radiation sensing device 22 is inserted into the retention assembly 38 such that the surface 30d of the radiation sensing device 22 is aligned with the second surface 36d of the retention assembly 38. During insertion of the sensing device holder 24 into the patient's mouth, the sensing device holder 24 is inserted with the retention guides 50, 52 first going into the mouth followed by the front surface 46a of the bite block 40, with the back surface 46b following. The retention assembly 38 and bite block 40 pass over the teeth of the patient and the support member 36, the retention guides 50, 52 and the radiation sensing device 22 are in the interior of the mouth. When the bite block 40 is correctly positioned proximate to the anterior teeth, the patient bites down onto the bite block 40 with his/her anterior teeth to securely hold the sensing device holder 24 in place. The bridge 42 extends outwardly from the teeth and through the lips of the patient. The mount 44 and rod 28 are exterior to the lips. The ring guide 26 overlaps the lips of the patient and the first surface 64a of the ring guide 26 faces the lips of the patient.

To take a bitewing right side image as shown in FIG. 19, the ring guide 26 is positioned such that the first surface 64a faces and overlaps the right cheek of the patient and the first connecting portion 66 extends forwardly of the lips of the patient. When looking at the patient from the right side, the second connecting portion 68 is at the ten o'clock position and the first connecting portion 66 is at the three o'clock position. The rod 28 is attached to the first connecting portion 66 and to the sensing device holder 24. The support member 36 overlaps the opening 70, and the back side 36b faces the first surface 64a of the ring guide 26. The radiation sensing device 22 is inserted into the retention assembly 38. During insertion of the sensing device holder 24 into the patient's mouth, the sensing device holder 24 is inserted with the leading retention guide 50 first going into the mouth followed by the leading end surface 44 of the bite block 40, with the trailing retention guide 52 and the trailing end 46f of the bite block 40 following. The retention assembly 38 and bite block 40 pass over the teeth of the patient and the support member 36, the retention guides 50, 52 and the radiation sensing device 22 are in the interior of the mouth. When the bite block 40 is correctly positioned proximate to the right bitewing teeth, the patient bites down onto the bite block 40 with his/her anterior teeth to securely hold the sensing device holder 24 in place. The bridge 42 extends outwardly from the teeth and through the lips of the patient. The mount 44 and rod 28 are exterior to the lips. The ring guide 26 overlaps the lips and part of the right cheek of the patient and the first surface 64a of the ring guide 26 faces the lips and part of the right cheek of the patient.

To take a bitewing left side image as shown in FIG. 20, the ring guide 26 is positioned such that the second surface 64b faces and overlaps the left cheek of the patient and the first connecting portion 66 extends forwardly of the lips of the patient. When looking at the patient from the left side, the second connecting portion 68 is at the two o'clock position and the first connecting portion 66 is at the nine o'clock position. The rod 28 is attached to the first connecting portion 66 and to the sensing device holder 24. The support member 36 overlaps the opening 70, and the back side 36b faces the second surface 64b of the ring guide 26. The radiation sensing device 22 is inserted into the retention assembly 38. During insertion of the sensing device holder 24 into the patient's mouth, the sensing device holder 24 is inserted with the leading retention guide 50 first going into the mouth followed by the leading end surface 44 of the bite block 40, with the trailing retention guide 52 and the trailing end 46f of the bite block 40 following. The retention assembly 38 and bite block 40 pass over the teeth of the patient and the support member 36, the retention guides 50, 52 and the radiation sensing device 22 are in the interior of the mouth. When the bite block 40 is correctly positioned proximate to the left bitewing teeth, the patient bites down onto the bite block 40 with his/her anterior teeth to securely hold the sensing device holder 24 in place. The bridge 42 extends outwardly from the teeth and through the lips of the patient. The mount 44 and rod 28 are exterior to the lips. The ring guide 26 overlaps the lips and part of the left cheek of the patient and the second surface 64b of the ring guide 26 faces the lips and part of the left cheek of the patient.

Therefore, the system 20 is very versatile. The system 20 can be easily changed to accommodate all images needed of the patient's mouth.

While particular embodiments are illustrated in and described with respect to the drawings, it is envisioned that those skilled in the art may devise various modifications without departing from the spirit and scope of the appended claims. It will therefore be appreciated that the scope of the disclosure and the appended claims is not limited to the specific embodiments illustrated in and discussed with respect to the drawings and that modifications and other embodiments are intended to be included within the scope of the disclosure and appended drawings. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the disclosure and the appended claims.

What is claimed is:

1. A system configured for use during a medical procedure comprising:
at least one sensing device holder comprising a support member and a retention assembly extending from the support member, the support member configured to be held between the teeth of a patient and the retention guide assembly configured to hold an associated radiation sensing device;
a ring guide comprising
a ring having opposite first and second surfaces, a central opening extending between the first and second surfaces and defining an axial centerline of the ring guide, the ring defining first and second axes which are perpendicular to each other, which pass through the axial centerline, and which are co-planar with the first and second surfaces,
a first connecting portion extending outwardly from the ring, the first connecting portion having a first channel extending therethrough, the channel intersecting the first axis, the first channel being parallel to the axial centerline, the first channel being offset from the second axis by a predetermined first distance along the first axis,
a second connecting portion extending outwardly from the ring and being spaced from the first connecting portion by an obtuse angle, the second connecting portion having a second channel extending therethrough, the second channel being offset from the first axis by a predetermined distance along the second axis, the second channel being parallel to the axial centerline, the second channel being offset from the second axis by a predetermined second distance along the first axis; and
a rod configured to be attached to either one of the first connecting portion and the second connecting portion, the rod detatchably connecting the sensing device holder to the ring guide.

2. The system of claim 1, wherein the rod is slidably attached to either one of the first connecting portion and the second connecting portion.

3. The system of claim 1, wherein the first distance and the second distance are equal.

4. The system of claim 1, wherein the first channel has a non-circular profile, and the rod has a mating non-circular cross-section.

5. The system of claim 1, wherein the second channel has a non-circular profile, and the rod has a mating non-circular cross-section.

6. The system of claim 1, wherein the first and second channels have a non-circular profile, and the rod has a mating non-circular cross-section.

7. The system of claim 1, wherein the ring is circular.

8. The system of claim 1, wherein two sensing device holders are provided, wherein a first one of the sensing device holders is configured to hold an associated radiation sensing device in a vertical position, and a second one of the sensing device holders is configured to hold an associated radiation sensing device in a horizontal position.

9. The system of claim 1, wherein the rod is linear and has a non-circular cross-section.

10. The system of claim 1, wherein the rod is formed of a rigid material.

11. The system of claim 8, wherein the rod is formed of one of plastic and metal.

12. The system of claim 1, wherein the sensing device holder comprises
an elongated support member having a front side and a rear side;
a retention assembly attached to the front side of the support member;
a bite block attached to and extending from the back side of the support member;
a bridge extending from the bite block;
a mount extending from the bridge, the mount having a single channel therethrough which is configured to have an associated rod attached thereto.

13. The system of claim 12, wherein the rod is insertable into the channel.

14. The system of claim 12, wherein the rod is insertable through the channel.

15. The system of claim 12, wherein the sensing device holder is a one-piece unit which is integrally formed.

16. The system of claim 12, wherein the sensing device holder has a recess therein.

17. A sensing device holder comprising:
an elongated support member having a front side and a rear side;
a retention assembly attached to the front side of the support member;
a bite block attached to and extending from the back side of the support member;
a bridge extending from the bite block;
a mount extending from the bridge, the mount having a single channel therethrough which is configured to have an associated rod attached thereto.

18. The sensing device holder of claim 17 in combination with a rod, the rod being insertable into the channel.

19. The sensing device holder of claim 17 in combination with a rod, the rod being insertable through the channel.

20. The sensing device holder of claim 17, wherein the sensing device holder is a one-piece unit which is integrally formed.
